(19) United States
(12) Patent Application Publication
(10) Pub. No.: US 2022/0378679 A1
(43) Pub. Date: Dec. 1, 2022

(54) COSMETIC PRODUCT COMPRISING A COSMETIC COMPOSITION CONTAINED IN AN APPLICATOR

(71) Applicant: The Procter & Gamble Company, Cincinnati, OH (US)

(72) Inventor: SHUHEI TANAKA, Singapore (SG)

(21) Appl. No.: 17/751,883

(22) Filed: May 24, 2022

Related U.S. Application Data
(60) Provisional application No. 63/195,044, filed on May 30, 2021.

(51) Int. Cl. A61K 8/60; A61K 8/891; A61Q 19/08

(52) U.S. Cl. CPC A61K 8/60; A61K 8/891; A61Q 19/08; A61K 2800/874; A61K 2800/30

(57) ABSTRACT

The present invention is directed to a cosmetic product comprising a cosmetic composition contained in an applicator, wherein the cosmetic composition comprises from about 2% to about 20% of an endothermic material selected from the group consisting of a sugar alcohol, a disaccharide, and mixtures thereof, wherein the endothermic material has an endothermic amount when dissolved in water of 5 cal/g or more, and wherein the applicator comprising: an application surface for applying the cosmetics composition wherein the application surface comprises a ceramic and/or metal; and a reservoir having an opening which fluidly connects the application surface to the reservoir. The cosmetic product of the present invention provides prolonged adequate cooling sensation during and after application, while providing moisturized feel.

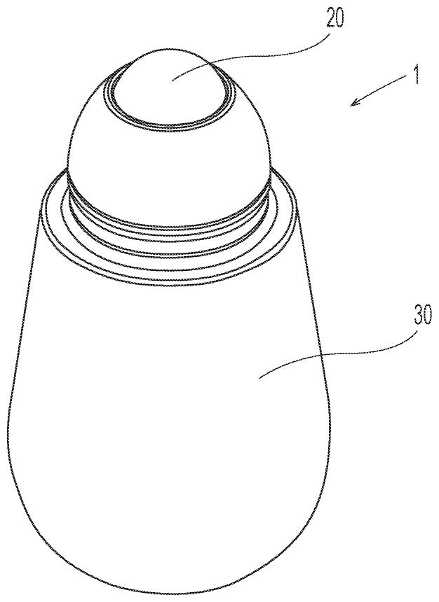

### FIELD OF THE INVENTION

The present invention is directed to a cosmetic product comprising a cosmetic composition contained in an applicator, wherein the cosmetic composition comprises from about 2% to about 20% of an endothermic material selected from the group consisting of a sugar alcohol, a disaccharide, and mixtures thereof, wherein the endothermic material has an endothermic amount when dissolved in water of 5 cal/g or more, and wherein the applicator comprising: an application surface for applying the cosmetics composition wherein the application surface comprises a ceramic and/or metal; and a reservoir having an opening which fluidly connects the application surface to the reservoir. The cosmetic product of the present invention provides prolonged adequate cooling sensation during and after application, while providing moisturized feel.

### BACKGROUND OF THE INVENTION

Mammalian keratinous tissue, particularly human skin, is subjected to a variety of insults by both extrinsic and intrinsic factors. Such extrinsic factors include ultraviolet radiation, environmental pollution, wind, heat, infrared radiation, low humidity, harsh surfactants, abrasives, etc. Intrinsic factors, on the other hand, include chronological aging and other biochemical changes from within the skin. Whether extrinsic or intrinsic, these factors result in visible signs of skin damage. Typical skin damage in aging or damaged skin include fine lines, wrinkling, hyperpigmentation, sallowness, sagging, dark under-eye circles, puffy eyes, enlarged pores, diminished rate of turnover, and abnormal desquamation or exfoliation. Additional damage incurred as a result of both external and internal factors includes visible dead skin i.e., flaking, scaling, dryness, and roughness.

Currently, there are a number of personal care products that are available to consumers, which are directed toward improving the health and physical appearance of keratinous tissues such as the skin, hair, and nails. The majority of these products are directed to delaying, minimizing or even eliminating skin wrinkling, spots, and other histological changes typically associated with the aging of skin or environmental damage to human skin. Consumers prefer topically applied products since they are not only effective, but also safe and pleasant to use.

Some cosmetic compositions are provided with applicators to deliver the above benefits, for example, to the targeted area of skin. For example, eye care products.

Some of cosmetic products which provide cooling sensation during product application are known. Cooling sensation can be provided from the cosmetic composition. Alternatively, cooling sensation can be provided by an applicator, for example, from heat transfer from skin to its metallic application surface.

Menthol and its derivatives are known to provide cooling sensation when contained in cosmetic compositions. However, such cooling sensation may be too strong to some users and/or to users who apply such composition to sensitive area of skin such as facial skin around eyes. Ethanol are also known to provide cooling sensation and/or refresh feeling when contained in cosmetic compositions. However, there may exists difficulties to provide prolonged cooling sensation and/or refresh feeling from ethanol. Also, ethanol may also provide dry feel and/or reduced moisturized feel.

Based on the foregoing, there exists a need for cosmetic products which provide prolonged adequate cooling sensation during and after application, while providing moisturized feel.

### SUMMARY OF THE INVENTION

The present invention is directed to a cosmetic product comprising a cosmetic composition contained in an applicator, wherein the cosmetic composition comprises from about 2% to about 20% of an endothermic material selected from the group consisting of a sugar alcohol, a disaccharide, and mixtures thereof, wherein the endothermic material has an endothermic amount when dissolved in water of 5 cal/g or more, and wherein the applicator comprising: an application surface for applying the cosmetics composition wherein the application surface comprises a ceramic and/or metal; and a reservoir having an opening which fluidly connects the application surface to the reservoir.

It has now been surprisingly found that, by the use of the specific endothermic material together with the specific application surface, the cosmetic product of the present invention provides prolonged adequate cooling sensation during and after application, while providing moisturized feel. The cosmetic product of the present invention provides such prolonged adequate cooling sensation at ambient temperatures, without the need for refrigeration or outside cooling.

Differently from menthol which provide cooling sensation by sensorial receptor activation (TRPM8), the specific endothermic material provides adequate cooling sensation when combined with the specific application surface. Also, differently from ethanol which provide cooling sensation due to evaporation of ethanol, the specific endothermic material provides prolonged cooling sensation especially when combined with the specific application surface.

Endothermic material is known to provide cooling sensation when dissolving in water. It is not to bound to the theory, but it is believed that: the specific endothermic material in the present composition is solidified at least partially during application due to evaporation of aqueous carrier in the composition, then, the solidified endothermic material dissolves in skin moisture, sweat, and/or remaining aqueous carrier from the composition on skin surface. It is also believed that the specific application surface especially that being rolling ball enhances the evaporation of aqueous carrier of the composition, thus, can provide cooling sensation at earlier timing of the application.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

The Figure is an applicator.

### DETAILED DESCRIPTION OF THE INVENTION

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all ingredient percentages are based on the weight of the cosmetic composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive and combinable to form narrower ranges not explicitly disclosed. For example, delineated upper and lower range limits are interchangeable to create further ranges.

The compositions of the present invention can comprise, consist essentially of, or consist of, the essential components as well as optional ingredients described herein. As used herein, “consisting essentially of means that the composition or component may only include additional ingredients that do not materially alter the basic and novel characteristics of the claimed composition or method. As used in the description and the appended claims, the singular forms “a”, “an”, and “the” are intended to include the plural forms as well, unless the context clearly indicates otherwise.

### Definitions

“About” modifies a particular value by referring to a range equal to plus or minus twenty percent (+1-20%) or less (e.g., less than 15%, 10%, or even less than 5%) of the stated value.

“Apply” or “application”, as used in reference to a composition, means to apply or spread the compositions of the present invention onto a human skin surface such as the epidermis.

“Derivative,” herein, means amide, ether, ester, amino, carboxyl, acetyl, and/or alcohol derivatives of a given compound. “Effective amount” means an amount of a compound or composition sufficient to significantly induce a positive benefit to keratinous tissue over the course of a treatment period. The positive benefit may be a health, appearance, and/or feel benefit, including, independently or in combination, the benefits disclosed herein.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration, food additives, and materials used in non-cosmetic consumer products including over-the-counter medications.

“Cosmetic composition” means a composition comprising a cosmetic agent. Examples of cosmetic compositions include color cosmetics (e.g., foundations, lipsticks, concealers, and mascaras), skin care compositions (e.g., moisturizers and sunscreens), personal care compositions (e.g., rinse-off and leave on body washes and soaps), hair care compositions (e.g., shampoos and conditioners).

“Skin care” means regulating and/or improving a skin condition (e.g., skin health, appearance, or texture/feel). Some nonlimiting examples of improving a skin condition include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism).

“Skin care composition” means a composition that includes a skin care active and regulates and/or improves skin condition.

“Synergy,” and variations thereof, means that the effect provided by a combination of two or more materials is more than the additive effect expected for these materials.

“Treatment period,” as used herein, means the length of time and/or frequency that a material or composition is applied to a target skin surface.

### COSMETIC PRODUCT

The cosmetic product of the present invention comprises a cosmetic composition contained in an applicator, wherein the cosmetic composition comprises from about 2% to about 20% of an endothermic material selected from the group consisting of a sugar alcohol, a disaccharide, and mixtures thereof, wherein the endothermic material has an endothermic amount when dissolved in water of 5 cal/g or more, and wherein the applicator comprising: an application surface for applying the cosmetics composition wherein the application surface comprises a ceramic and/or metal; and a reservoir having an opening which fluidly connects the application surface to the reservoir.

Preferably, the cosmetic product is an eye care product from which the cosmetic composition is applied to facial skins around eyes.

Details of the cosmetic composition and the applicator are described below.

### APPLICATOR

The figure shows applicator 1 comprising application surface 20 adapted for applying the cosmetic composition wherein application surface 20 having a ceramic and/or metal outer surface; and a reservoir 30 having an opening which fluidly connects the application surface 20 to the reservoir 30. Reservoir 30 is adapted for storing a cosmetic composition. Preferably, applicator surface 20 is a rotating ball having a ceramic and/or metal outer surface that can provide a cooling sensation.

### COSMETIC COMPOSITION

The cosmetic composition useful herein comprises from about 2% to about 20% of an endothermic material selected from the group consisting of a sugar alcohol, a disaccharide, and mixtures thereof, wherein the endothermic material has an endothermic amount when dissolved in water of 5 cal/g or more.

The composition has a viscosity of preferably from about 100 cP to about 15,000 cP, more preferably from about 1000 cP to about 12,000 cP, still more preferably from about 4000 cP to about 9,000 cP, in view of at least one of the followings: providing fresh penetration feel during application; easiness to apply to skin; and easiness to dispense from the applicator.

Preferably, the cosmetic composition useful herein is substantially free of menthol and its derivatives, in view of providing adequate cooling sensation. In the present invention, “the composition being substantially free of menthol and its derivatives” means that: the composition is free of menthol and its derivatives; or, if the composition contains menthol and its derivatives, the level of such menthol and its derivatives is very low. In the present invention, the total level of such menthol and its derivatives is, if included, 0.05% or less, preferably 0.01% or less, more preferably 0% by weight of the composition.

Preferably, the composition of the present invention contains limited amount of ethanol in view of providing moisturized feel, for example, at a level of up to about 5%, more preferably up to about 3%, still more preferably up to about 1% by weight of the composition, even more preferably 0%, i.e., the composition is free of ethanol.

The cosmetic compositions herein are intended for topical application to human skin. The compositions herein may optionally include one or more additional skin actives or other ingredients of the type commonly included in topical cosmetic compositions.

The cosmetic compositions herein may be cosmetic compositions, pharmaceutical compositions, or cosmeceutical compositions, and may be provided in various product forms, including, but not limited to, solutions, suspensions, lotions, gels, toners, cleansing liquid washes hydrogels, film-forming products, and the like. In some instances, the composition form may follow from the particular dermatologically acceptable carrier chosen. For example, the composition (and carrier) may be provided in the form of an emulsion (e.g., water-in-oil, oil-in-water, or water-in-oil-in water) or an aqueous dispersion. Preferably, the cosmetic composition of the present invention is in the form of an oil-in-water emulsion.

### ENDOTHERMIC MATERIAL

The endothermic materials useful herein are those selected from the group consisting of a sugar alcohol, a disaccharide, and mixtures thereof, wherein the endothermic material has an endothermic amount when dissolved in water of 5 cal/g or more, preferably 15 cal/g or more, more preferably 30 cal/g or more.

Such sugar alcohols include, for example, xylitol (having an endothermic amount when dissolved in water of about 36.6 cal/g), erythritol (having an endothermic amount when dissolved in water of about 43 cal/g), sorbitol (having an endothermic amount when dissolved in water of about 26.6 cal/g).

Such disaccharide includes, for example, trehalose (having an endothermic amount when dissolved in water of about 12.1 cal/g).

Among such endothermic materials, preferred are sugar alcohols, more preferred are sugar alcohols selected from the group consisting of xylitol, erythritol, sorbitol and mixtures thereof, still more preferred is xylitol.

The endothermic material is contained in the composition at a level by weight of the composition of from about 2% to about 10%, preferably from about 2.2% to about 7%, more preferably from about 2.5% to 5%.

### SILICONE OIL

Preferably, in view of reducing sticky and/or tacky feel from the specific endothermic materials of the present invention while maintaining moisturized feel also from the specific endothermic materials of the present invention, the cosmetic composition further comprises a silicone oil which is a mixture of: a high molecular weight silicone having a viscosity of about 100,000 mPa·s or more at 25° C.; and a silicone solvent having a viscosity of from about 1 mPa·s to about 5,000 mPa·s, preferably from about 2 mPa·s to about 2,000 mPa·s, more preferably from about 2 mPa·s to about 1,000 mPa·s.

Preferably, the weight ratio of the high molecular weight silicone to the silicone solvent is from about 1:2 to about 1:100, more preferably from about 1:5 to about 1:70.

Preferably, the weight ratio of the high molecular weight silicone to the endothermic material is from about 1:10 to about 1:100, more preferably 1:15 to about 1:70, still more preferably from about 1:20 to about 1:50.

Preferably, the weight ratio of the silicone oil to the endothermic material is from about 1:20 to about 5:1, more preferably from about 1:10 to about 3:1, still more preferably from about 1:5 to about 3:1.

The viscosity can be measured by means of a glass capillary viscometer as set forth in Dow Corning Corporate Test Method CTM0004, Jul. 20, 1970.

High molecular weight silicone useful herein include, for example, polydimethylsiloxane which is also known as dimethicone, dimethiconol, polydiethylsiloxane, and polymethylphenylsiloxane (phenyl dimethicone). Dimethicone and dimethiconol are especially preferred.

The high molecular weight silicone useful herein also include a silicone gum. The term “silicone gum”, as used herein, means a polyorganosiloxane material having a viscosity at 25° C. of greater than or equal to 1,000,000 centistokes and/or having a mass molecular weight in excess of about 200,000, generally from about 200,000 to about 4,000,000 or to about 1,000,000. It is recognized that the silicone gums described herein can also have some overlap with the above-disclosed silicone compounds. This overlap is not intended as a limitation on any of these materials. Specific examples include polydimethylsiloxane (dimethicone), dimethiconol, poly (dimethylsiloxane methylvinylsiloxane) copolymer, poly (dimethylsiloxane diphenylsiloxane methylvinylsiloxane) copolymer and mixtures thereof. The silicone gums are often available, for example, as a mixture with silicone compounds having a lower viscosity. Such mixtures include, for example: a blend of 12% of dimethiconol gum and 88% of dimethicone (5cSt) available from Dow Corning with a tradename DC1503 having viscosity of 1500 cP; a blend of dimethiconol gum and cyclopentasiloxane available from Dow Corning with a tradename DC1501 having a viscosity of 6000 cP; a blend of dimethicone having a viscosity of 18,000,000 mPa·s and dimethicone having a viscosity of 200 mPa·s available from GE Toshiba; and a blend of dimethicone having a viscosity of 18,000,000 mPa·s and cyclopentasiloxane available from GE Toshiba.

Silicone solvent useful herein include, for example, polydimethylsiloxane which is also known as dimethicone, polydiethylsiloxane, and polymethylphenylsiloxane. Dimethicone is especially preferred. Preferably, the silicone solvent is non-volatile.

Dermatologically Acceptable Carrier

The compositions disclosed herein may include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In some instances, the dermatologically acceptable carrier is in the form of an emulsion. The emulsion may have a continuous aqueous phase (e.g., an oil-in-water or water-in-oil-in-water emulsion) or a continuous oil phase (e.g., water-in-oil or oil-in-water-in-oil emulsion). The oil phase of the present invention may comprise silicone oils, non-silicone oils such as hydrocarbon oils, esters, ethers, and mixtures thereof. The aqueous phase typically comprises water and water-soluble ingredients (e.g., water-soluble moisturizing agents, conditioning agents, anti-microbials, humectants and/or other skin care actives). However, in some instances, the aqueous phase may comprise components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s).

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 30% by weight of composition (e.g., 1% to 20%, 2% to 15%, 3% to 12%, 4% to 10%, or even 5% to 8%) to help avoid some of the undesirable feel effects of oily compositions. The oil phase may include one or more volatile and/or non-volatile oils (e.g., botanical oils, silicone oils, and/or hydrocarbon oils).

### Aqueous Carrier

The composition preferably comprises an aqueous carrier. Preferably, the aqueous carrier is substantially water. Deionized water is preferably used. Water from natural sources including mineral cations can also be used, depending on the desired characteristic of the product.

In one embodiment, the aqueous carrier can be present at a level of from about 50% to about 99%, preferably from about 60% to about 98%, more preferably from about 70% to about 98%, still more preferably from about 80% to about 95%, by weight of the composition.

### Skin Conditioning Agent

Optionally, the composition of the present invention can further comprise a skin conditioning agent. These agents may be selected from humectants and emollients. The amount of skin-condition agent may range from about 1% to about 50%, preferably from about 2% to about 40%, more preferably from about 5% to about 30%, by weight of the composition.

Humectants are polyhydric alcohols intended for moisturizing, reducing scaling and stimulating removal of built-up scale from the skin. Typical polyhydric alcohols include polyalkylene glycols and more preferably alkylene polyols and their derivatives. Illustrative are propylene glycol, dipropylene glycol, polypropylene glycol, polyethylene glycol, sorbitol, hydroxypropyl sorbitol, hexylene glycol, 1,3-butylene glycol, 1,2,6-hexanetriol, ethoxylated glycerin, propoxylated glycerin and mixtures thereof. Most preferably the humectant is glycerin.

When the conditioning agent is an emollient it may be selected from hydrocarbons, fatty acids, fatty alcohols and esters.

### Fatty Alcohol

The compositions herein may include a fatty alcohol. Fatty alcohols refer to high-molecular-weight, straight-chain primary alcohols that have the general structure: where n=8 to 32.

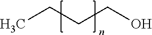

Fatty alcohols may be natural or synthetic, saturated or unsaturated, branched or straight-chain. Some nonlimiting examples of fatty alcohols commonly used in cosmetic compositions include caprylic, capryl, lauryl, myristyl, cetyl, stearyl, and behenyl alcohols. The fatty alcohols herein may be referred to generically by the number of carbon atoms in the molecule. For example, a “C12 alcohol” refers to an alcohol that has 12 carbon atoms in its chain (i.e., dodecanol).

The fatty alcohol may be included in the compositions herein at 0.0001% to 15% (e.g., 0.0002% to 10%, 0.001% to 15%, 0.025% to 10%, 0.05% to 7%, 0.05% to 5%, or even 0.1% to 3%) by weight of the composition.

### Whitening Agents

The present compositions may contain a whitening agent. The whitening agent useful herein refers to active ingredients that not only alter the appearance of the skin, but further improve hyperpigmentation as compared to pre-treatment. Useful whitening agents useful herein include ascorbic acid compounds, vitamin B 3 compounds, azelaic acid, butyl hydroxy anisole, gallic acid and its derivatives, hydroquinoine, kojic acid, arbutin, mulberry extract, tetrahydrocurcumin, and mixtures thereof. Use of combinations of whitening agents is also believed to be advantageous in that they may provide whitening benefit through different mechanisms.

When used, the compositions preferably contain from about 0.1% to about 10%, more preferably from about 0.2% to about 5%, by weight of the composition, of a whitening agent.

Ascorbic acid compounds are useful whitening agents and ascorbyl glucoside is a preferred derivative of the ascorbic acid compounds.

### Conditioning Agents

The compositions herein may include 0.1% to 50% by weight of a conditioning agent (e.g., 0.5% to 30%, 1% to 20%, or even 2% to 15%). Adding a conditioning agent can help provide the composition with desirable feel properties (e.g., a silky, lubricious feel upon application). Some non-limiting examples of conditioning agents include, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof.

Particularly suitable examples of conditioning agents include non-volatile silicone fluids other than those described above under the title “SILICONE OIL”, such as dimethicone copolyol, dimethylpolysiloxane, diethylpolysiloxane, mixed C1-30 alkyl polysiloxanes, phenyl dimethicone, dimethiconol, dimethicone, dimethiconol, silicone crosspolymers, and combinations thereof. Dimethicone may be especially suitable, since some consumers associate the feel properties provided by certain dimethicone fluids with good moisturization.

### Thickening Agents

The compositions of the present invention, in some embodiments, may further include one or more thickening agents.

Nonlimiting classes of thickening agents include those selected from the following: carboxylic acid polymers, crosslinked polyacrylate polymers, polyacrylamide polymers, polysaccharides and gums.

When present, the composition preferably includes from about 0.01% to about 5%, more preferably from about 0.1% to about 4%, and still more preferably from about 0.1% to about 3%, by weight of the composition of the thickening agent.

### Emulsifiers

When the composition is in the form of an emulsion, it may contain an emulsifier.

Emulsifiers may be nonionic, anionic, cationic, or zwitterionic. Some non-limiting examples of emulsifiers are disclosed in U.S. Pat. Nos. 3,755,560, 4,421,769, U.S. Publication No. 2006/0275237 and McCutcheon's Detergents and Emulsifiers, North American Edition, pages 317-324 (1986). Suitable examples of emulsifiers include non-ionic stearic acid derivative emulsifiers such as Polysorbate-20, PEG-100 stearate, Steareth-2, Steareth-21, glycereth-25 pyrrolidonecarboxylic acid isostearate and combinations of these.

### Other Optional Ingredients

Compositions herein may include one or more optional ingredients known for use in topical cosmetic compositions, provided that the optional components do not unacceptably alter the desired benefits of the composition. In some instances, it may be desirable to select cosmetic actives that function via different biological pathways so that the actives do not interfere with one another. When the composition is in the form of an emulsion, the additional ingredients should not introduce instability into the emulsion (e.g., syneresis). For example, it may be desirable to select optional ingredients that do not form complexes with other ingredients in the composition, especially pH sensitive ingredients like vitamin B3 compounds, salicylates and peptides.

The additional ingredients should be suitable for use in contact with human skin tissue without undue toxicity, incompatibility, instability, allergic response, and the like. The optional components, when present, may be included at an amount of about 0.001% to 50% (e.g., 0.01% to 40%, 0.1% to 30%, 0.5% to 20%, or 1% to 10%), by weight of the composition. Some non-limiting examples of additional ingredients include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunscreen agents, sunless tanning agents, lubricants, anti-acne agents, anti-cellulite agents, chelating agents, anti-wrinkle actives, anti-atrophy actives, phytosterols and/or plant hormones, N-acyl amino acid compounds, antimicrobials, and antifungals. Some particularly suitable examples of additional ingredient include one or more skin care actives selected from the group consisting of vitamin B3 compounds (e.g., niacinamide), n-acyl amino acids (e.g., undecylenoyl phenylalanine), vitamin E compounds (e.g., tocopheryl acetate), palmitoylated dipeptides (e.g., palmitoyl-lysine-threonine), palmitoylated pentapeptides (e.g., palmitoyl-lysine-threonine-threonine-lysine-serine), vitamin A compounds (e.g., retinol and retinyl propionate), and combinations thereof. Other non-limiting examples of optional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/0092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

Sucrose esters may be used herein. Such sucrose ester can be a blend of two or more sucrose esters, wherein the two or more sucrose esters are present at a ratio of any one sucrose ester to another of 1:10 to 1:1 (e.g., 1:7, 1:5, 1:3, or 1:2). In some instances, the sucrose ester may be a blend of sucrose laurate and sucrose dilaurate, wherein sucrose laurate is present at 50% to 80%, by weight of the sucrose ester, and the sucrose dilaurate is present at 20% to 45%, by weight of the sucrose ester. Alternatively, the sucrose ester may be a blend of sucrose laurate, sucrose dilaurate and sucrose trilaurate, wherein the sucrose dilaurate is present at 35% or more, by weight of the sucrose ester. A particularly suitable example of a sucrose ester for use herein is Sucrose

Dilaurate BC10034 available from BASF.

### Method of Use of the Cosmetic Composition

The method of use herein includes identifying a target portion of skin on a person in need of treatment and applying the composition to the target portion of skin over the course of a treatment period. The target portion of skin may be on a facial skin surface such as the forehead, perioral, chin, periorbital, nose, and/or cheek) or another part of the body (e.g., hands, arms, legs, back, chest). The person in need of treatment is one whose skin exhibits signs of oxidative stress, such as fine lines, wrinkles, hyperpigmentation, uneven skin tone, and/or other visible skin features typically associated with aging. In some instances, the target portion of skin may not exhibit a visible sign of skin aging, but a user (e.g., a relatively young user) may still wish to target such an area of skin, if it is one that typically develops such issues as a person ages. In this way, the present method may be used as a preventative measure to delay the onset of visible signs of skin aging.

The composition may be applied to a target portion of skin and, if desired, to the surrounding skin at least once a day, twice a day, or on a more frequent daily basis, during a treatment period. When applied twice daily, the first and second applications are separated by at least 1 to 12 hours. Typically, the composition is applied in the morning and/or in the evening before bed. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The step of applying the composition may be accomplished by localized application. In reference to application of the composition, the terms “localized”, “local”, or “locally” mean that the composition is delivered to the targeted area (e.g., a wrinkle or line) while minimizing delivery to skin surfaces where treatment is not desired. The composition may be applied and lightly massaged into an area of skin. The form of the composition or the dermatologically acceptable carrier should be selected to facilitate localized application. While certain embodiments herein contemplate applying a composition locally to an area, it will be appreciated that compositions herein can be applied broadly to one or more skin surfaces. In certain embodiments, the compositions herein may be used as part of a multi-step beauty regimen, wherein the present composition may be applied before and/or after one or more other compositions.

### COMBINATIONS

A. A cosmetic product comprising a cosmetic composition contained in an applicator,
   a. wherein the cosmetic composition comprises from about 2% to about 20% of an endothermic material selected from the group consisting of a sugar alcohol, a disaccharide, and mixtures thereof, wherein the endothermic material has an endothermic amount when dissolved in water of 5 cal/g or more, and
   b. wherein the applicator comprising: an application surface for applying the cosmetics composition wherein the application surface comprises a ceramic and/or metal; and a reservoir having an opening which fluidly connects the application surface to the reservoir.
B. The cosmetic product of the preceding feature, wherein the endothermic material is a sugar alcohol, preferably a sugar alcohol selected from the group consisting of xylitol, erythritol, sorbitol and mixtures thereof, more preferably xylitol.
C. The cosmetic product of any of the preceding features, wherein the composition comprises from about 2.2% to about 7% of the endothermic material.
D. The cosmetic product of any of the preceding features, wherein the composition further comprises a silicone oil which is a mixture of: a high molecular weight silicone having a viscosity of from about 100,000 mPa·s to about 30,000,000 mPa·s; and a silicone solvent having a viscosity of from about 1 mPa·s to 5,000 mPa·s.
E. The cosmetic product of any of the preceding features, wherein the silicone solvent having a viscosity of from about 2 mPa·s to about 2,000 mPa·s.
F. The cosmetic product of any of the preceding features, wherein the weight ratio of the high molecular weight silicone to the silicone solvent is from about 1:2 to about 1:100.
G. The cosmetic product of any of the preceding features, wherein the weight ratio of the high molecular weight silicone to the endothermic material is from about 1:10 to about 1:100, preferably from about 1:15 to about 1:70, more preferably from about 1:20 to about 1:50.
H. The cosmetic product of any of the preceding features, wherein the composition is substantially free of menthol and its derivatives.
I. The cosmetic product of any of the preceding features, wherein the applicator surface is a rotating ball.
J. The cosmetic product of any of the preceding features, wherein the cosmetic product is an eye care product.

### Examples

Cosmetic Compositions (Wt %)

| | Comp.1 | Comp.2 | Comp.3 | Comp.4 | Comp. 5 | Comp.6 |
|---|---|---|---|---|---|---|
| Water Purified | q.s. to 100% | q.s. to 100% | q.s. to 100% | q.s. to 100% | q.s. to 100% | q.s. to 100% |
| Glycerin | 5.0 | 7.0 | 5.0 | 8.0 | 10.0 | 10.0 |
| Xylitol | 3.0 | | | 2.5 | 5.0 | 10.0 |
| Trehalose | | 3.0 | | 0.5 | | |
| Erythritol | | | 3.0 | | | |
| D-panthenol | 0.5 | 0.5 | 0.5 | 1.0 | 0.5 | 1.0 |
| Hexyldecanol | | | | 5.0 | 1.0 | |
| Pentylene Glycol | | 2.0 | 2.0 | 3.0 | 3.0 | 3.0 |
| 1,3-Butylene Glycol | | 5.0 | 5.0 | 10.0 | 2.0 | 2.0 |
| Hexylene Glycol | | | | 0.75 | 0.75 | |
| Hexandiol | | | | 0.25 | | 0.5 |
| DC1503 *1 | 0.75 | | 1.5 | 1.0 | 2.0 | 3.0 |
| DC1501 *2 | | 0.5 | | | | |
| PMX-200 Silicone Fluid | 1.75 | | 1.0 | | 2.0 | 4.0 |
| 5cSt *3 | | | | | | |
| PMX-200 Silicone Fluid | 1.75 | | | 2.0 | | |
| 50cSt *4 | | | | | | |
| PMX-200 Silicone Fluid | 0.75 | 1.0 | | | | |
| 10,000cSt *5 | | | | | | |
| PEG-20 *6 | | | | | 1.0 | |
| PEG-32 *7 | | | | | 1.0 | 1.0 |
| KSG-16 *8 | | | | | | 2.0 |
| Sepigel 305 *9 | | 0.6 | 0.6 | 0.6 | 1.0 | |
| Simulgel EG *10 | | 0.5 | | | | |
| Ultrez 20 * 11 | | | | 0.25 | | 0.25 |
| Ultrez 21 *12 | | | | | 0.1 | |
| Pemulen TR-2 *13 | | | | | 0.15 | |
| Xanthan gum | 0.05 | 0.05 | | 0.05 | 0.1 | 0.2 |
| Sepimax Zen *14 | 1.0 | 0.9 | 1.2 | | | |
| Niacinamide | 5.0 | 5.0 | 5.0 | 5.0 | 5.0 | 5.0 |
| Isopropyl Isostearate | 2.0 | | | | 2.0 | |
| Inositol | 0.3 | 0.3 | 0.5 | 0.3 | | |
| Nylon Powder SP-500 *15 | | | 1.5 | | | 1.5 |
| KSP-100*16 | 4.0 | | | | | 1.5 |
| Tospearl 145A *17 | | | | | 2.0 | 3.0 |
| Eldew PS-203 *18 | 1.5 | 1.5 | 1.5 | 1.0 | 2.0 | |
| Aminomethyl Propanol | | | | 0.15 | 0.15 | 0.15 |
| Preservative | 0.25 | 0.25 | 0.25 | 0.20 | 0.10 | 0.10 |
| Perfume | 0.05 | 0.05 | 0.05 | 0.05 | 0.05 | 0.05 |

Details of the Ingredients Used in the Above Table

| | | |
|---|---|---|
| *1 | DC1503 | Dimethicone (5 cSt, Solvent) and Dimethiconol |
| *2 | DC1501 | Cyclopentasiloxane (Solvent) and Dimethiconol |
| *3 | PMX-200 Silicone | Dimethicone 5 cSt |
| | Fluid 5 cSt | |
| *4 | PMX-200 Silicone | Dimethicone 50 cSt |
| | Fluid 50 cSt | |
| *5 | PMX-200 Silicone | Dimethicone 10,000 cST |
| | Fluid 10,000 cSt | |
| *6 | PEG-20 | having MW of 1000 |
| *7 | PEG-32 | having MW of 1540 |
| *8 | KSG-16 | INCI: Dimethicone/Vinyl Dimethicone |
| | | Crosspolymer and Dimethicone |
| *9 | Sepigel 305 | Containing 40% of Polyacrylamide as active. |
| | | INCI: Polyacrylamide & Water & C13-14 |
| | | Isoparaffin & Lauleth-7 |
| *10 | Simulgel EG | Containing 37.5% of Sodium Acrylate/Sodium |
| | | Acryloyldimethyl Taurate Copolymer as active |
| *11 | Ultrez 20 | INCI: Acrylates/C10-30 Alkyl Acrylate |
| | | Crosspolymer |
| *12 | Ultrez 21 | INCI: Acrylates/C10-30 Alkyl Acrylate |
| | | Crosspolymer |
| *13 | Pemulen TR-2 | INCI: Acrylates/C10-30 Alkyl Acrylate |
| | | Crosspolymer |
| *14 | Sepimax Zen | INCI: Polyacrylate crosspolymer-6 |
| *15 | Nylon Powder | Nylon-12 |
| | SP-500 | |
| *16 | KSP-100 | INCI: Vinyl Dimethicone/Methicone |
| | | Silsesquioxane Crosspolymer |
| *17 | Tospearl 145A | INCI: Polymethylsilsesquioxane |
| *18 | Eldew PS-203 | INCI: Phytosteryl/Octyldodecyl Lauroyl |
| | | Glutamate |

Applicator Applicator 1: Applicator used herein comprises an application surface for applying the cosmetics composition wherein the application surface is a rotating ball comprising a ceramic; and a reservoir having an opening which fluidly connects the application surface to the reservoir.

Applicator 2: Applicator used herein comprises an application surface for applying the cosmetics composition wherein the application surface is a rotating ball comprising a metal; and a reservoir having an opening which fluidly connects the application surface to the reservoir.

Cosmetic Product

| | Ex. 1 | Ex. 2 | Ex. 3 | Ex. 4 | Ex. 5 | Ex. 6 |
|---|---|---|---|---|---|---|
| Composition | Comp. 1 | Comp. 2 | Comp. 3 | Comp. 4 | Comp. 5 | Comp. 6 |
| Applicator | Applicator 1 | Applicator 2 | Applicator 1 | Applicator 1 | Applicator 2 | Applicator 1 |

Cosmetic product examples Ex. 1 through Ex. 6 are the cosmetic products of the present invention, which are suitably used as eye care products. The cosmetic products of the present invention provide prolonged adequate cooling sensation during and after application, while providing moisturized feel.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic product comprising a cosmetic composition contained in an applicator, wherein the cosmetic composition comprises from about 2% to about 20% of an endothermic material selected from the group consisting of a sugar alcohol, a disaccharide, and mixtures thereof, wherein the endothermic material has an endothermic amount when dissolved in water of 5 cal/g or more, and
wherein the applicator comprising: an application surface for applying the cosmetics composition wherein the application surface comprises a ceramic and/or metal; and a reservoir having an opening which fluidly connects the application surface to the reservoir.

2. The cosmetic product of claim 1, wherein the endothermic material comprises one or more sugar alcohols.

3. The cosmetic product of claim 2, wherein the one or more sugar alcohols comprise xylitol, erythritol, sorbitol, or a combination thereof.

4. The cosmetic product of claim 3, wherein the sugar alcohol is xylitol.

5. The cosmetic product of claim 1, wherein the composition comprises from about 2.2% to about 7% of the endothermic material.

6. The cosmetic product of claim 1, wherein the composition further comprises a silicone oil which is a mixture of a high molecular weight silicone comprising a viscosity of about 100,000 mPa·s or more and a silicone solvent comprising a viscosity of from about 1 mPa·s to 5,000 mPa·s.

7. The cosmetic product of claim 6, wherein the silicone solvent comprising a viscosity of from about 2 mPa·s to about 2,000 mPa·s.

8. The cosmetic product of claim 6, wherein the weight ratio of the high molecular weight silicone to the silicone solvent is from about 1:2 to about 1:100.

9. The cosmetic product of claim 6, wherein the weight ratio of the high molecular weight silicone to the endothermic material is from about 1:10 to about 1:100.

10. The cosmetic product of claim 9, wherein the weight ratio of the high molecular weight silicone to the endothermic material is from about 1:15 to about 1:70.

11. The cosmetic product of claim 10, wherein the weight ratio of the high molecular weight silicone to the endothermic material is from about 1:20 to about 1:50.

12. The cosmetic product of claim 1, wherein the composition is substantially free of menthol and its derivatives.

13. The cosmetic product claim 1, wherein the applicator surface is a rotating ball.

14. The cosmetic product of any of the preceding claim, wherein the cosmetic product is an eye care product.
